(19) United States
(12) Patent Application Publication
(10) Pub. No.: US 2023/0172520 A1
(43) Pub. Date: Jun. 8, 2023

(54) AUTOMATIC ELECTRO-ANATOMICAL (EA) DATA POINTS SELECTION

(71) Applicant: BIOSENSE WEBSTER (ISRAEL) LTD., Yokneam (IL)

(72) Inventors: Benjamin Cohen, Haifa (IL); Natan Sharon Katz, Atlit (IL); Vladimir Dvorkin, Kiryat Motzkin (IL); Lior Zar, Poria Illit (IL)

(21) Appl. No.: 17/542,739

(22) Filed: Dec. 6, 2021

(51) Int. Cl. A61B 5/367; A61B 5/00; A61B 5/363; A61B 5/361

(52) U.S. Cl. CPC A61B 5/367; A61B 5/7221; A61B 5/7475; A61B 5/363; A61B 5/361

(57) ABSTRACT

A method for configuring an electroanatomical (EA) mapping procedure, the method includes receiving user input including a type of arrhythmia to be diagnosed. A confidence level is received for data points acquired in a heart of a patient in the EA mapping procedure. One or more data collection parameters for the data points are automatically set based on the type of arrhythmia and the confidence level. An EA map is constructed and presented, the EP map including at least some of the acquired data points, in accordance with the automatically-set data collection parameters.

### FIELD OF THE DISCLOSURE

This disclosure relates generally to analysis of intracardiac electroanatomical (EA) signals, and specifically to automatic selection of EA data points to generate an EA map.

### BACKGROUND OF THE DISCLOSURE

Computer aided analysis of intracardiac signals was previously suggested in the patent literature. For example, U.S. Patent Application Publication No. 2018/0125575 describes devices and methods for tissue lesion assessment and/or creation based on dielectric properties. In some embodiments, one or more probing frequencies are delivered via electrodes including an electrode in proximity to a tissue (for example, myocardial tissue). Measured dielectric properties (such as impedance properties), optionally together with other known and/or estimated tissue characteristics, are used to determine the lesion state of the tissue. In some embodiments, a developing lesion state is monitored during treatment formation of a lesion (for example, ablation of heart tissue to alter electrical transmission characteristics).

### BRIEF DESCRIPTION OF THE DRAWINGS

The present disclosure will be more fully understood from the following detailed description of the embodiments thereof, taken together with the drawings, in which:

FIG. 1 is a schematic, pictorial illustration of a system for electroanatomical (EA) mapping, in accordance with an exemplary embodiment of the present disclosure;

FIGS. 2A and 2B are schematic illustrations of histograms of collected data with algorithm-controlled limit settings for an aggregate number of data points below and above an algorithm threshold, respectively, according to an exemplary embodiment of the present disclosure;

FIG. 3 is a flow chart that schematically illustrates a method and algorithm to perform automatic EA data point selection using the histograms of FIGS. 2, according to an exemplary embodiment of the present disclosure; and

FIG. 4 is a flow chart that schematically illustrates a method and algorithm for automatic retroactive EA data point selection using the algorithm of FIG. 3, according to an exemplary embodiment of the present disclosure.

### DETAILED DESCRIPTION OF EMBODIMENTS

### Overview

In a patient suffering from cardiac arrythmia, electrical activity impulses may be generated at a pathological tissue region and/or follow a pathologic path in cardiac tissue. In an electroanatomical (EA) study, one of the goals of the physician is to identify such pathological regions and/or paths of the electrical activity, e.g., by inserting a multi-electrode catheter into the heart of the patient and measuring intracardiac ECG signals via the catheter. Typically, the catheter is threaded through blood vessels to the heart to measure the electrical activity in the endocardium.

To measure the electrical activity, some electrodes of the catheter are brought into contact with heart tissue to acquire an electrical potential difference between the electrodes and a ground, such as a Wilson Central Terminal ground, or between two electrodes of the catheter. An electrical potential difference between a catheter electrode and the ground is called the unipolar signal of the electrode. An electrical potential difference between two electrodes of the catheter is called the bipolar signal between the electrodes.

In order to understand the paths followed by the impulse of the electrical activity during an EA study, the physician needs to know the time at which the impulse passed under each of the electrodes. To this end, a set of data points is acquired, the data points comprising (i) measured locations on a wall tissue of a cardiac chamber and (ii) respective electrical activation signals from which the EP mapping system can produce an EA map. One example of an EA map that is useful in diagnosing arrhythmia is an EA timing diagram map, called a local activation time (LAT) map, of regions of the cardiac chamber wall tissue.

To generate an LAT map, a processor may have to analyze intracardiac ECG signals collected at various points in the heart, called hereinafter electrograms (EGM), to identify an activation in each signal (i.e., in a waveform of an EGM), and to annotate the activation and calculate an LAT value.

An annotation time represents the time when the cardiac impulse passed a specific cardiac tissue location, as measured by the catheter. Since different EGM signals are collected at different times, a gated measurement technique is sometimes used to align the signals collected at different times. In the domain of cardiac electrophysiology, the “gate” used for the gated measurement is called the reference annotation. Examples include the R peak of the QRS signal of the body surface ECG, an activation detected inside the coronary sinus, activations detected at the high-right-atrium, or a more sophisticated method fed by multiple signals may be used as the reference annotation. The LAT value is defined as the difference between the mapping annotation time and the reference annotation time.

Typically, every heartbeat can be used to update the EA map. The operator needs to define criteria for the system in order to collect only the “good” data points acquired during each heartbeat. Those data points are applied to the EA map and color coded according to their EA properties. Based on the map coloring, the physician can correctly diagnose the arrhythmia. However, it is difficult to both choose the best criteria to provide a good distribution of data points and also select only the “good” points that contribute to the understanding of the map.

To obtain an EA map of diagnostic value, an operator of an EA mapping system needs to define EA parameters (e.g., cycle length, stability of catheter, stability of LAT values, among other parameters) for the system to collect only “relevant” data points. Those points are applied to the EA map and contribute to its coloring. Based on the coloring, the physician is able to correctly diagnose the disease.

The challenge is, on the one hand, to select the best criteria that will provide a good distribution of points, and, on the other hand, to select only the “relevant” points that will contribute to the understanding of the map. Sometimes, during a procedure, the parameters must be adjusted, e.g., according to the changes occurring in the patient. Setting the parameters correctly requires skill and time.

Embodiments of the present disclosure that are described hereafter provide a technique and algorithm configured to automatically perform EA parameter selection. Moreover, some embodiments enable a user to generate other maps, or a same map with different settings, that include initially rejected data points. To this end in these embodiments, a processor collects all acquired data points and stores them in memory to serve as point cloud from which the maps can be generated.

In some embodiments, the operator initially selects the arrythmia type to be tracked, chooses the level of confidence (termed hereinafter “confidence level”) desirable for the data points and then, based on those inputs and the collected data, the algorithm automatically sets the collection parameters for the data points.

The confidence level can be an adjustable level (e.g., confidence level that is obtained automatically by the algorithm, starting with low confidence and the algorithm raising the bar as more high-quality points are collected). The confidence level can alternatively be a fixed, required confidence level (e.g., a level set in advance as a target minimal confidence level to reach). In one embodiment, the confidence level has an initial value that is subsequently adjusted as more data points are acquired. In another embodiment, the confidence level is a required confidence level set in advance by the algorithm or the user.

For example, based on the confidence level (adjustable or fixed), the processor determines how stringent to set the limits of at least some of the acquisition parameters. The processor constructs and presents an EA map, comprising at least some of the acquired data points, in accordance with the automatically-set data collection parameters.

For example, if the operator wants to map flutter, the algorithm sets relevant acquisition parameters to collect only relevant data points, as follows:

1. Cycle length (CL) is steady within a given tolerance and thus the system will capture 95% of data points with the “same” CL value (i.e., will drop acquired data points with CL off tolerance).

2. LAT stability and position stability parameters should be set very high to allow high-quality data points to be captured (for example, by setting level of confidence above a minimal value, such as half, with the level ranging between zero and one).

3. As the EA map should be uniform over the chamber, the map should be high density (e.g., data points in isolated positions are discarded).

4. Signal-to-noise ratio (SNR) of data points is set to be above a given threshold.

As another example, if the operator wants to map Atrial Fibrillation (AFib), the algorithm sets relevant acquisition parameters that include LAT stability and LAT regional consistency.

As yet another example, if the operator wants to map Ventricle Tachycardia (VT) Fibrillation, the algorithm sets relevant acquisition parameters of ECG pattern matching, CL stability, position stability, and LAT stability.

In an embodiment, as the processor aggregates data points, the processor generates a histogram for each parameter, and maintains only data points, for example, that fall within a predefined range. Initially, when the number of acquired data points is low, the processor uses a wide predefined range. As the number of acquired data points increases, the processor narrows the range, meaning that the processor applies stricter limits to determine a relevant portion of the data points that make the histogram.

In other embodiments, instead of the operator initially selecting the parameters for collecting the data points, the disclosed algorithm is initially run to have the system start aggregating all the data points captured by a catheter for a case. Once enough data points have been collected, the algorithm automatically analyzes the data and statistically selects the best combination of acquisition parameters to collect further data points.

For example, the processor may identify changes in the type of arrhythmia during treatment, (e.g., an Atrial Fibrillation reduced to flutter by ablation), and adjust the acquisition parameter settings in real time to generate a relevant EA map to a current condition of the patient. To this end, the algorithm may tighten the constraints over the LAT values to be within narrower limits.

By providing the aforementioned automated processes, an accuracy of an EA map can improved at a lower workload by an operator of the invasive cardiac mapping system.

### System Description

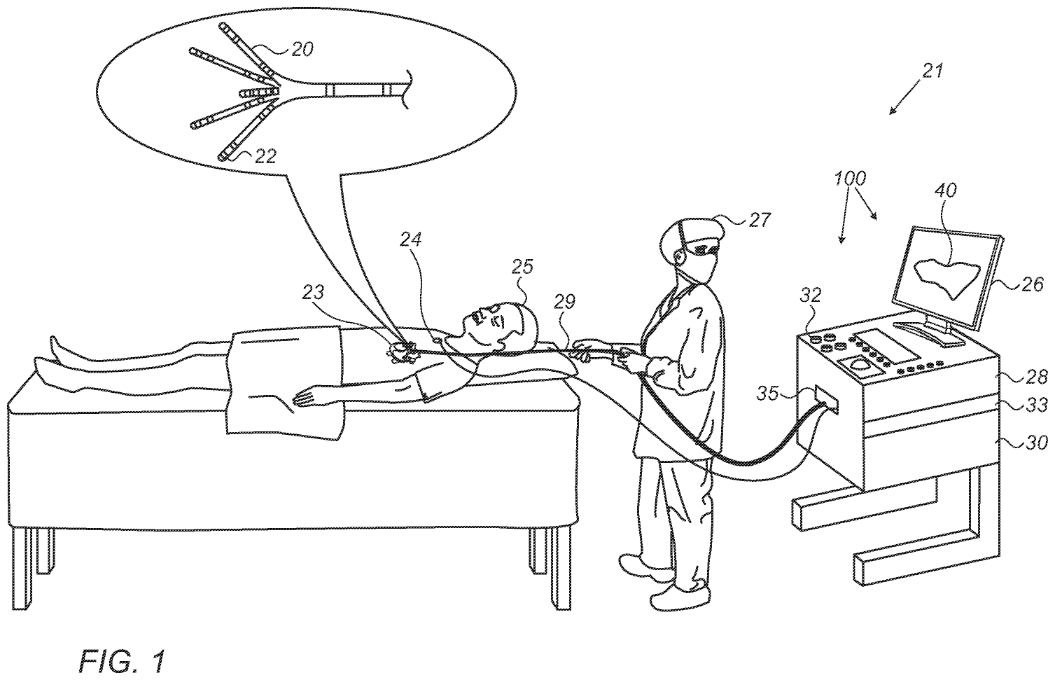

FIG. 1 is a schematic, pictorial illustration of a system 21 for electroanatomical (EA) mapping, in accordance with an embodiment of the present disclosure. FIG. 1 depicts a physician 27 using a Pentaray® EA mapping catheter 29 to perform an EA mapping of a heart 23 of a patient 25. Catheter 29 comprises, at its distal end, one or more arms 20, which may be mechanically flexible, each of which is coupled with one or more electrodes 22. During the mapping procedure, electrodes 22 acquire and/or inject unipolar and/or bipolar signals from and/or to the tissue of heart 23.

A processor 28 in a console 30 receives these signals via an electrical interface 35, and uses information contained in these signals, and the disclosed algorithm for automatic selection of data points, to generate an EA map 40 from the data points, such as a LAT map or a CL map. Processor 28 stores the data points and the map in a memory 33, which also holds the required disclosed software. During and/or following the procedure, processor 28 may display EA map 40 on a display 26. User controls 32 of a user interface 100 enable physician 27 to communicate with processor 28 to lock portions of EA map 40 to prevent further updating, as described above. Controls 32 may include, for example, a trackball and control knobs, as well as a keyboard. Other elements of user interface 100 may include touch screen functionality of display 26.

During the procedure, a tracking system is used to track the respective locations of sensing electrodes 22, such that each of the signals may be associated with the location at which the signal was acquired. For example, the Active Current Location (ACL) system, made by Biosense-Webster (Irvine, Calif.), which is described in U.S. Pat. No. 8,456,182, whose disclosure is incorporated herein by reference, may be used. In the ACL system, a processor estimates the respective locations of the electrodes based on impedances measured between each of the sensing electrodes 22, and a plurality of surface electrodes 24 that are coupled to the skin of patient 25. For example, three surface electrodes 24 may be coupled to the patient's chest, and another three surface electrodes may be coupled to the patient's back. For ease of illustration, only one surface electrode is shown in FIG. 1. Electric currents are passed between electrodes 22 inside heart 23 of the patient and surface electrodes 24. Processor 28 calculates an estimated location of all electrodes 22 within the patient's heart based on the ratios between the resulting current amplitudes measured at surface electrodes 24 (or between the impedances implied by these amplitudes) and the known positions of electrodes 24 on the patient's body. The processor may thus associate any given impedance signal received from electrodes 22 with the location at which the signal was acquired.

The example illustration shown in FIG. 1 is chosen purely for the sake of conceptual clarity. Other tracking methods can be used, such as those based on measuring voltage signals. Other types of sensing catheters, such as the Lasso® Catheter (produced by Biosense Webster) may equivalently be employed. Contact with tissue sensors may be fitted at the distal end of EA mapping catheter 29. As noted above, other types of electrodes, such as those used for ablation, may be utilized in a similar way and fitted to electrodes 22 for acquiring the needed position data. In an embodiment, measurements of one or more electrodes 22 may be discarded if their physical contact quality is indicated as poor, and the measurements of other electrodes may be regarded as valid if their contact quality is indicated as sufficient. Thus, an ablation electrode used for collecting position data is regarded, in this case, as a sensing electrode. In an optional embodiment, processor 28 is further configured to indicate the quality of physical contact between each of the electrodes 22 and an inner surface of the cardiac chamber during measurement.

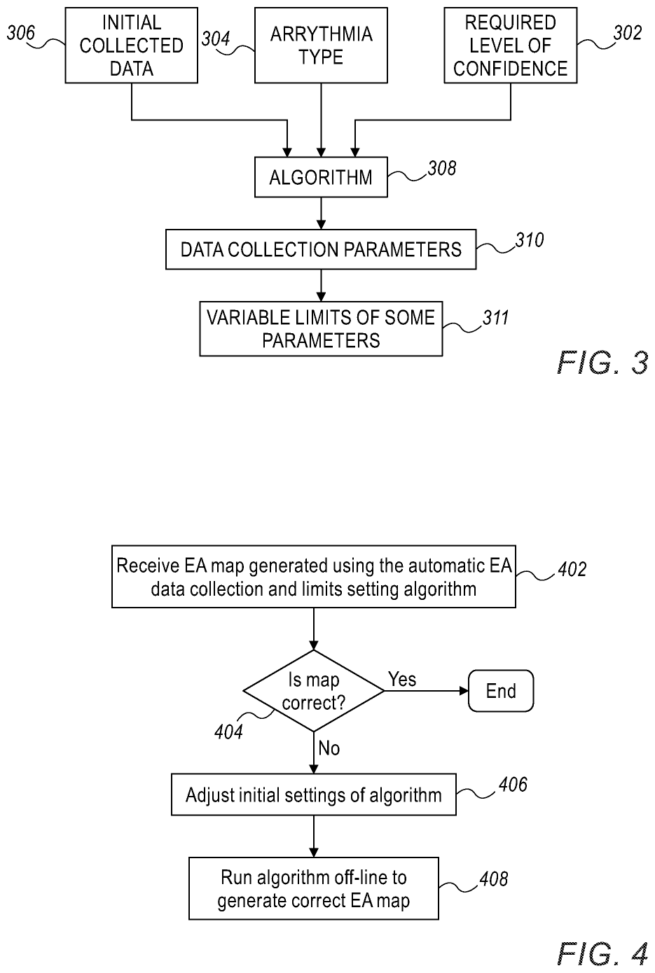

Processor 28 typically comprises a general-purpose computer with software programmed to carry out the functions described herein. In particular, processor 28 runs a dedicated algorithm as disclosed herein, including in FIG. 3, that enables processor 28 to perform the disclosed steps, as further described below. The software may be downloaded to the computer in electronic form, over a network, for example, or it may, alternatively or additionally, be provided and/or stored on non-transitory tangible media, such as magnetic, optical, or electronic memory.

Automatic Ea Data Points Selection Using Histograms

During EA mapping the aggregate number of acquired data points typically increases from hundreds to well over 10,000. As noted above, the disclosed algorithm has the system start aggregating all the data points captured by a catheter for a case. Initially, the algorithm uses most or all acquired data points by having wide predefined limits on acquisition parameters. As the number of data points increases, the algorithm gradually narrows at least some of the limits, for example, according to an actual level of confidence vs. preset target level, and/or to meet conditions set by a cascade of increasing thresholds for number of acquired data points.

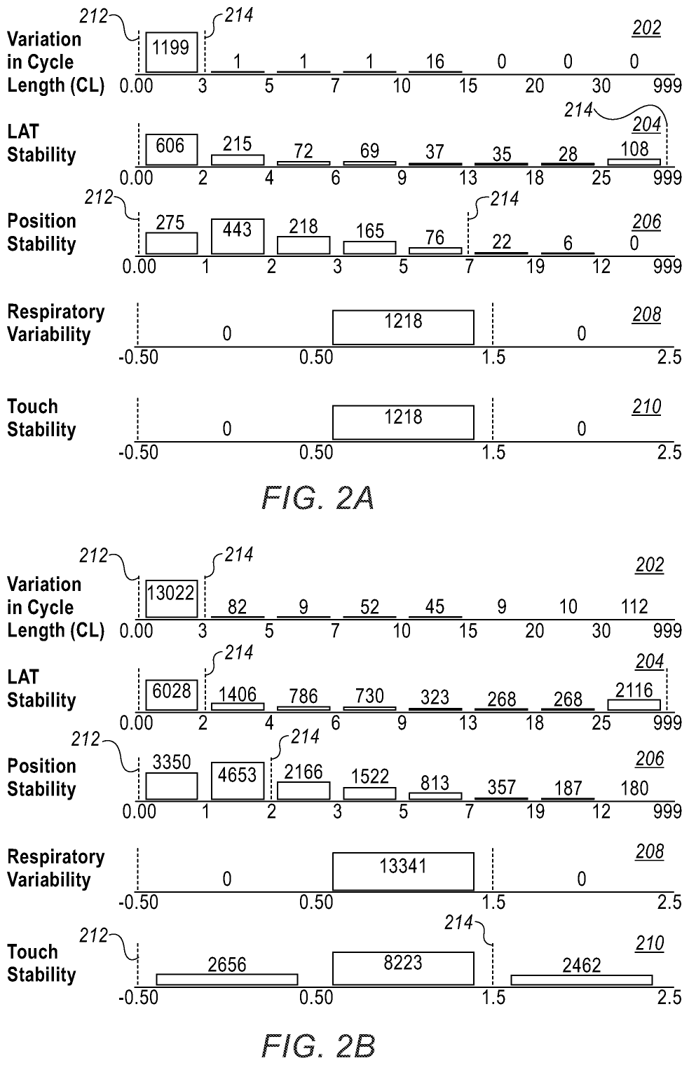

For example, as the number of data points increases beyond a first threshold (e.g., 2,000 data points), the processor may apply the disclosed algorithm to start to maintain only data points that meet a set of criteria, which is visualized in FIG. 2 as limits over histograms of collected data.

FIGS. 2A and 2B are schematic illustrations of histograms 202-210 of collected data with the algorithm-controlled lower and upper limits 212 and 214, respectively, for a low aggregate number of data points (e.g., below the first threshold) and high aggregate number (e.g., >10,000), respectively, according to an embodiment of the present disclosure.

The data points are collected during an EA mapping of a flutter type of cardiac rhythm problem.

As seen in FIG. 2A, when the aggregate number of data points is below threshold (e.g., 1218<2000), the algorithm uses all available data points to construct an EA map, by setting limits 214 to maximum, to capture the entire histogram (e.g., of LAT stability), or almost in entirety (e.g., position stability).

When the aggregate number of data points goes above threshold (e.g., above 2000), the processor gradually narrows more and more (e.g., lowers according to cascade of thresholds) limits 214 to the minima shown in FIG. 2B.

As seen in FIG. 2B, by the time the aggregate number of data points is far above threshold (e.g., 13,341>2000), the algorithm uses only part of data points (e.g., parts of histograms) to construct an EA map, using tighter limits 214 on, for example, LAT stability and position stability histograms.

As noted above, the processor saves the entire data point cloud. Thus, the processor can run an automatic selection of EA data points to generate a new EA map offline. For example, in case a reference signal is deemed wrong or malfunctioned, the processor may use another reference signal and rerun the automatic process to generate a more relevant EA map.

The example illustrations shown in FIGS. 2A and 2B are chosen purely for the sake of conceptual clarity. For example, in types of arrhythmia other than flutter, different parameters may be increasingly limited by the algorithm.

Method for Automatic Ea Data Point Selection

FIG. 3 is a flow chart that schematically describes a method and an algorithm to perform automatic EA data point selection using the histograms of FIGS. 2, according to an embodiment of the present disclosure. The shown process includes an algorithm input step 302 where a user sets a required level of confidence, and a step 304, where the user sets a type of arrhythmia to map. The type of arrhythmia is generally known to the user, for example, from medical history of the patient. Based on the level of confidence and type of arrhythmia, algorithm 308 sets:
a) The data collection parameters 310 (such as LAT, bipolar potential, position, etc.)
b) Variable limits 311 (e.g., limits 212 and 214 of FIG. 2) for at least part of the parameters, to maintain only conforming data points based, for example, on the aggregate number 306 of acquired data points, as shown in the varying limits on the LAT stability and position stability histograms 204 and 206, respectively, of FIGS. 2A and 2B.

The example flow chart shown in FIG. 3 is chosen purely for the sake of conceptual clarity. The present embodiment may also comprise additional steps of the algorithm, such as receiving ECG signals in parallel to the catheter-acquired signals. This and other possible steps are omitted from the disclosure herein purposely in order to provide a more simplified flow chart.

Automatic Retroactive Ea Data Point Selection

As noted above, the processor saves the entire data point cloud to enable generation of alternative or additional EA maps using the disclosed algorithm.

FIG. 4 is a flow chart that schematically illustrates a method and algorithm for automatic retroactive EA data point selection using the algorithm of FIG. 3, according to an embodiment of the present disclosure. The process begins at a receiving step 402, at which a user receives (e.g., reviews on a display) an EA map that was automatically generated using the algorithm of FIG. 3.

At a decision step 404, if the user finds the map correct, or relevant, the process ends. If, on the other hand, the user finds the map to be flawed, sub-optimal, or irrelevant, the user can generate an improved map by rerunning the algorithm.

To this end, the user adjusts, or changes, initial algorithm settings, at a setting adjustment step 406. For example, if the user finds that a reference electrode was not functioning well (either a body surface electrode or a catheter electrode), the user can change the reference electrode. As another example, if the user finds that the level of confidence was not optimal, the user may adjust the level of confidence.

Finally, at an offline running step 408, the user reruns the algorithm, and the processor mimics the real-time EA data point selection and EA map generation.

An embodiment of the present disclosure that was described above provides a method for configuring an electroanatomical (EA) mapping procedure, the method including receiving user input including a type of arrhythmia to be diagnosed. A confidence level is received for data points acquired in a heart of a patient in the EA mapping procedure. One or more data collection parameters for the data points are automatically set based on the type of arrhythmia and the confidence level. An EA map is constructed and presented, the EP map including at least some of the acquired data points, in accordance with the automatically-set data collection parameters.

In some embodiments, the method further includes adjusting the received confidence level in response to acquisition of additional data points.

In some embodiments, the confidence level is a required confidence level set in advance.

In an embodiment, the data collection parameters include at least one parameter type selected from a group of types consisting of a cardiac cycle length, a position of a catheter acquiring the data points, a contact force of the catheter, a Local Activation Time (LAT), a bipolar potential and a unipolar potential.

In some embodiments, the type of arrhythmia is one of ventricle tachycardia, flutter and atrial fibrillation.

In an embodiment, setting the one or more data collection parameters includes selecting at least one of the data collection parameters based the type of arrhythmia.

In another embodiment, the method further includes automatically adjusting a limit on values of a given data collection parameter, and including in the EA map only the data points whose values are within the limit.

In yet another embodiment, automatically adjusting the limit includes constructing a histogram of the values of the given data collection parameter, and deriving the limit from the histogram.

In some embodiments, the method further includes constructing and presenting the EA map off-line, using retroactively-set data collection parameters.

There is additionally provided, in accordance with another embodiment of the present disclosure, an apparatus for configuring an electroanatomical (EA) mapping procedure, the apparatus including a user interface (100) and a processor (28). The user interface is configured to receive user input including a type of arrhythmia to be diagnosed. The processor is configured to (i) receive a confidence level for data points acquired in a heart of a patient in the EA mapping procedure, (ii) automatically set one or more data collection parameters for the data points, based on the type of arrhythmia and the confidence level, and (iii) construct and present an EA map, including at least some of the acquired data points, in accordance with the automatically-set data collection parameters.

Although the embodiments described herein mainly address cardiac EA mapping systems, the methods and systems described herein can also be used, mutatis mutandis, in any medical application that needs to acquire neural activations, such as electroencephalograms (EEG).

It will be thus appreciated that the embodiments described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A method for configuring an electroanatomical (EA) mapping procedure, the method comprising:
receiving user input comprising a type of arrhythmia to be diagnosed;
receiving a confidence level for data points acquired in a heart of a patient in the EA mapping procedure;
automatically setting one or more data collection parameters for the data points, based on the type of arrhythmia and the confidence level;
constructing and presenting an EA map, comprising at least some of the acquired data points, in accordance with the automatically-set data collection parameters.

2. The method according to claim 1, and comprising adjusting the received confidence level in response to acquisition of additional data points.

3. The method according to claim 1, wherein the confidence level is a required confidence level set in advance.

4. The method according to claim 1, wherein the data collection parameters comprise at least one parameter type selected from a group of types consisting of a cardiac cycle length, a position of a catheter acquiring the data points, a contact force of the catheter, a Local Activation Time (LAT), a bipolar potential and a unipolar potential.

5. The method according to claim 1, wherein the type of arrhythmia is one of ventricle tachycardia, flutter and atrial fibrillation.

6. The method according to claim 1, wherein setting the one or more data collection parameters comprises selecting at least one of the data collection parameters based the type of arrhythmia.

7. The method according to claim 1, further comprising automatically adjusting a limit on values of a given data collection parameter, and including in the EA map only the data points whose values are within the limit.

8. The method according to claim 7, wherein automatically adjusting the limit comprises constructing a histogram of the values of the given data collection parameter, and deriving the limit from the histogram.

9. The method according to step 1, and comprising constructing and presenting the EA map off-line, using retroactively-set data collection parameters.

10. An apparatus for configuring an electroanatomical (EA) mapping procedure, the apparatus comprising:
an interface, configured to receive user input comprising a type of arrhythmia to be diagnosed; and
a processor, configured to:
receive a confidence level for data points acquired in a heart of a patient in the EA mapping procedure;
automatically set one or more data collection parameters for the data points, based on the type of arrhythmia and the confidence level; and
construct and present an EA map, comprising at least some of the acquired data points, in accordance with the automatically-set data collection parameters.

11. The apparatus according to claim 10, wherein the processor is configured to adjust the received confidence level in response to acquisition of additional data points.

12. The method according to claim 10, wherein the confidence level is a required confidence level set in advance.

13. The apparatus according to claim 10, wherein the data collection parameters comprise at least one parameter type selected from a group of types consisting of a cardiac cycle length, a position of a catheter acquiring the data points, a contact force of the catheter, a Local Activation Time (LAT), a bipolar potential and a unipolar potential.

14. The apparatus according to claim 10, wherein the type of arrhythmia is one of ventricle tachycardia, flutter and atrial fibrillation.

15. The apparatus according to claim 10, wherein the processor is configured to select at least one of the data collection parameters based the type of arrhythmia.

16. The apparatus according to claim 10, wherein the processor is further configured to automatically adjust a limit on values of a given data collection parameter, and to include in the EA map only the data points whose values are within the limit.

17. The apparatus according to claim 16, wherein the processor is configured to automatically adjust the limit by constructing a histogram of the values of the given data collection parameter, and deriving the limit from the histogram.

18. The apparatus according to step 10, wherein the processor is further configured to construct and present the EA map off-line, using retroactively-set data collection parameters.
